(19) United States
(12) Patent Application Publication
(10) Pub. No.: US 2022/0249789 A1
(43) Pub. Date: Aug. 11, 2022

(54) BALLOON HAVING A MULTI-LAYER WALL STRUCTURE FOR THE TISSUE-CONSERVING LOW-PRESSURE SEALING OF OPENINGS AND CAVITIES IN THE BODY OF A PATIENT, IN PARTICULAR IN THE CASE OF CYCLICALLY FLUCTUATING FILLING PRESSURE VALUES

(71) Applicant: Creative Balloons GmbH, Waghäusel (DE)

(72) Inventor: Fred Göbel, Speyer (DE)

(21) Appl. No.: 17/611,707

(22) PCT Filed: May 18, 2020
(86) PCT No.: PCT/IB20/54684

§ 371 Date: Nov. 16, 2021

(30) Foreign Application Priority Data
May 16, 2019 (DE) 10 2019 003 482.1

(51) Int. Cl. A61M 16/04; A61L 29/04

(52) U.S. Cl. CPC A61M 16/0443; A61M 2205/0216; A61L 29/049

(57) ABSTRACT

The invention relates to a multi-layer balloon film material in which one or more layers of an elastically deforming material is/are combined with one or more layers of a plastically deforming non-elastic material, wherein the non-elastic layer counteracts the straightening properties of the elastic layer in the case of planar folds or bends in the balloon film material. In particular, the cross-sectional area of eyelet- or channel-shaped structures which conducts secretions or fluids, which structures typically occur in the area of wrinkle-like invaginations of a residually sized balloon film in an organ lumen or a body cavity, can be reduced and, particularly in the case of cyclically changing filling pressures of the balloon, can be stabilized in such a way that an optimally sealing closure or tamponade effect is achieved across the largest possible filling pressure amplitudes.

### REFERENCE TO PENDING PRIOR PATENT APPLICATIONS

This patent application claims benefit of International (PCT) Patent Application No. PCT/IB2020/054684, filed 18 May 2020 by Creative Balloons GmbH for BALLOON HAVING A MULTI-LAYER WALL STRUCTURE FOR THE TISSUE-CONSERVING LOW-PRESSURE SEALING OF OPENINGS AND CAVITIES IN THE BODY OF A PATIENT, IN PARTICULAR IN THE CASE OF CYCLICALLY FLUCTUATING FILLING PRESSURE VALUES, which patent application, in turn, claims benefit of German Patent Application No. DE 10 2019 003 482.1, filed 16 May 2019.

The two (2) above-identified patent applications are hereby incorporated herein by reference.

### FIELD OF THE INVENTION

The invention is directed to a balloon-like structure for positioning within a cavity in the human or animal body, for example within a lumen or some other interior space, in particular as an integral part of a catheter, in such a way that the stated cavity on the one hand is filled as completely as possible, i.e., without residual space, but on the other hand largely maintains its shape or is not deformed by the balloon body.

### BACKGROUND OF THE INVENTION

Catheter applications in the body of a patient in many cases require balloon-like components that sealingly close off or tamponade in a space-filling manner a lumen or an interior space, wherein the sealing and/or tamponading function of the particular balloon is maintained even if the cross-sectional area of the lumen to be sealed, i.e., the volume of the space to be tamponaded, fluctuates intermittently or cyclically, for example, due to the physiological function of the particular structure or due to movements of the body. In addition, for balloon components that are placed in the body on a long-term basis, the force that is transmitted by the balloon to the adjacent tissue and structures should be in a range that preserves perfusion and rules out pressure-related lesions.

Basic approaches to the problem of efficient, tissue-compatible sealing of an organ or cavity have already been presented in EP 1 061 984 A1, with specific reference to sealing the trachea with respect to secretions of the pharynx. The special quality of the tracheal seal described therein is based essentially on the use of very thin-walled balloon films made of polyurethane (PUR). The circumference of the balloon (cuff) that is formed during manufacture exceeds the circumference of the tracheal portion to be sealed, which results in the formation of a typical invagination pattern of the balloon envelope when the residually dimensioned balloon is filled within the trachea, which is smaller relative to the balloon. The residual balloon material, i.e., the excess material along the circumference of the balloon, forms into radial invaginations, i.e., following the pattern of wheel spokes. At their blind end, i.e., the end pointing toward the center of the balloon, these invaginations form specific channel-like formations that follow the longitudinal axis of the balloon and allow the free flow of secretions or liquids. The formation of such invaginations is crucial for the low-pressure behavior and maintaining the perfusion of the adjacent structures. The principle of invagination of a residually dimensioned balloon envelope ensures that for closing off the particular lumen or space, the envelope does not have to be converted into the state of a force-intensive expansion, but, rather, may “fold” into the particular lumen or interior space in question of the patient without tension, which is possible even for balloon filling pressure values that only slightly exceed the particular local pressure. The particular shape and size of the lumen or other cavity of the patient are thus maintained.

EP 1 061 984 A1 describes, for a residually dimensioned balloon envelope made of PUR, a region having a certain wall thickness, namely, 5 to 20 μm, in which, for a balloon envelope placed in situ, channel-like formations arise whose inner diameter inhibits the free flow of a secretion or has a capillary, stasis-like effect on the secretion. The diameters of the tubules described in EP 1 061 984 A1 are less than 0.11 mm, advantageously less than 0.05 mm.

In addition to the material-specific design of the envelope itself, the diameter of the tubules that form in each case at the end position of the invaginations of the cuff envelope during the course of a tracheal ventilation situation is a function essentially of the particular filling pressure prevailing at that moment in the balloon. When the filling pressure decreases, the channel-like structures begin to expand, and the tubules, starting from the particular blind end of the invaginations, successively open toward the tracheal-side base or the opening of the invaginations. Upon a further drop in the filling pressure in the balloon, the invagination opens up toward the tracheal mucosa and goes into a configuration having an approximately U-shaped or also W-shaped pattern in the radial direction. As a function of the particular extent of the opening of the invaginations or the cross-sectional area of the tubules that effectively develops, this results in an effective sealing capability of the cuff at a certain point in time.

Within the scope of this type of sealing or tamponading balloon technology, a particular challenge is the sealing of organs or spaces whose internal pressure undergoes cyclical fluctuations, as is the case for the trachea or the esophagus, for example. Both structures are subjected to continuous, cyclical pressure fluctuations in the thorax which are generated by the patient's own breathing. For unassisted breathing as well as machine-assisted breathing of the patient, the particular generated thoracic pressures correspond to the filling pressure of the tracheal sealing cuff.

If the thoracic pressure prevailing in the thoracic cavity drops during inspiration by the patient, this transient decrease in pressure is transferred to the tracheal and esophageal walls, which in turn results in a pressure reduction in corresponding tracheally or esophageally positioned balloon structures. At the moment when pressure on the tracheal or esophageal wall is relieved, the cross-sectional areas of the channel-like formations at the end position expand synchronously, which over the phase of the expansion results in an enlarged passage for secretions and liquids, which may lead to a complete loss of the seal of the balloon and bolus-like aspiration.

The special dependency of the sealing efficiency of tracheal tube cuffs on the thoracic work of breathing by the patient is addressed, among other sources, by Badenhorst et al. (“Changes in cuff pressure during respiratory support,” C. H. Badenhorst, Critical Care Medicine 1987, 15; 4, 300-302). Badenhorst describes cuff pressure values for individual patients, which started at approximately 20 mbar and extended up to the subatmospheric range, and which thus largely followed the intrathoracic pressure values prevailing in the thorax of the breathing patient.

In the current literature on the sealing behavior of residually dimensioned tracheal tube cuffs, so-called high-volume, low-pressure cuffs, the particular capability of certain types of cuffs for ensuring an effective secretion seal is determined using static observation models. Thus, for example, in a study by Bassi et al. (“An in vitro study to assess determinant features associated with fluid sealing in the design of endotracheal tube cuffs and exerted tracheal pressures,” Bassi et al., Critical Care Medicine, 2013; 41:518-526), a rigid tube was intubated with various tracheal tubes, followed by installation of a water column above the tracheal tube cuff acted on by filling pressure. For conventional PVC-based cuff types, the leakage determined in the static model was already in the range of several milliliters per minute at an applied, recommended filling pressure of 30 mbar. Strictly PUR-based, very thin-walled cuff types still allowed a reliable sealing effect even when the filling pressure was reduced to 15 mbar. However, for filling pressures below 15 mbar, even PUR balloons having wall thicknesses of less than 20 μm showed an initial expansion of the secretion-conducting, channel-like formations, wherein, the same as for the PVC cuffs manufactured with much thicker walls, the cross sections of the tubules from the blind end of the particular invagination toward their base opened in a droplet shape and ultimately allowed more secretions and liquids to pass through.

In summary, static models are not able to accommodate the described cyclically fluctuating changes in the tracheal cross-sectional area as a scalable factor. Thus, they are unsuitable for depicting the clinically effective quality of a balloon-based seal in the trachea under pressure fluctuations.

### SUMMARY OF THE INVENTION

In order to improve the efficiency of sealing balloon components under the conditions of organ-synchronous cyclical fluctuations of the balloon filling pressure, the object of the invention is to provide novel types of cuff designs that ensure efficient sealing behavior even when the filling pressure of the balloon continuously fluctuates over a pressure amplitude ranging from 30 mbar to 5 mbar.

Within the scope of a generic balloon-like structure, this object is achieved in that the balloon is made of a multilayer balloon film material, at least one layer being made of an elastically deformable polyurethane (PUR) and at least one other layer being made of a nonelastic material such as polyvinyl chloride (PVC), wherein the at least one PUR layer is made of a thermoplastic PUR of a type having a water absorption of 5% or less according to DIN ISO 62, preferably having a water absorption of 2% or less according to DIN ISO 62.

The present invention thus describes approaches for improving the sealing behavior of soft film-like balloon bodies having a sealing and/or tamponading action, which are introduced into independently motile or also into passively motile organs or spaces in the body, where they may be permanently positioned, also and in particular when the particular internal pressure in the organ and/or the particular configuration of the space go(es) through changes intermittently, continuously, or in particular also in a cyclically fluctuating manner. The walls of the balloon bodies according to the invention have a specific, multilayer combined structure made up of nonelastically deforming and elastically deforming material layers.

The balloon film material includes one or more layers of an elastically deforming material together with one or more layers of a plastically deforming nonelastic material, wherein the nonelastic layer counteracts the straightening properties of the elastic layer in the event of planar folds or bends in the balloon film material.

For balloon components which have a corresponding multilayer structure and which form fold-like invaginations of the residual balloon envelope in situ upon placement and being acted on by filling pressure, the elastic straightening and opening properties of the cross-sectionally loop- or channel-like formations are reduced by the material composite which includes an nonelastic material, so that for intermittent or cyclical fluctuations of the balloon filling pressure, in relation to single-layer balloon envelopes made of elastic material, for example PUR, and having the same wall thickness, cross-sectional areas of the secretion-conducting cross-sectional areas at the end position that are smaller, and under the conditions of fluctuating filling pressure that are smaller overall and fluctuate to a lesser extent, form within the loop- and tubule-like structures. The behavior of such combinations, in which the wall thickness of the incorporated PUR layer is reduced to the smallest possible proportion of the overall wall thickness of the balloon envelope, is particularly advantageous.

If the balloon is formed in a blow molding process from a previously manufactured multilayer raw tubular material, the proportional PUR layer in the molding process has a stabilizing effect, and for balloon envelopes formed with particularly thin walls, also allows good symmetry of the balloon body, wherein the raw tube to be converted, when acted on by blowing pressure, successively transforms via a uniform spindle shape into a uniform spherical balloon shape, and subsequently expands into the particular blow mold and assumes its shape. The elastic properties of PUR allow, in addition to uniform symmetry, the qualitatively stable formation of balloon components having extremely low overall wall thicknesses in the range of 5 to 20 microns, for example, and in application also impart high mechanical load capacity, puncture resistance, and generally very good dimensional stability under transient and also long-term exceedances of the particular working filling pressures.

The combination according to the invention of one or more PVC-based material layers with a PUR layer that mechanically stabilizes the balloon body is also advantageous for reducing permeability effects of water molecules, which are typical for PUR. When a water-permeable PUR layer is joined to a PVC layer, which has much better barrier properties against polar substances than does PUR, in particular the condensation and accumulation of water in the balloon may be reduced.

In order to keep the overall wall thickness of a balloon film having a multilayer structure according to the invention as low as possible, i.e., in a preferred range of 5 to 30 microns, the invention proposes the use of types of PUR that are characterized by the lowest possible swelling tendency of the balloon wall due to the absorption of water molecules. Such swelling effects are known primarily for nonthermoplastic polyurethanes. The invention therefore preferably uses thermoplastic types of PUR having a water absorption of less than 4%, preferably less than 2%, according to DIN ISO 62 in the exposed aqueous environment, for example the types from the product line “Pellethane 2363” from Lubrizol Inc. or the product line “Elastollan 1100” from BASF AG.

### BRIEF DESCRIPTION OF THE DRAWINGS

Further features, properties, advantages, and effects on the basis of the invention result from the following description of several preferred embodiments of the invention, with reference to the drawings. In the drawings:

FIG. 1 shows a design according to the invention of a balloon envelope made up of two combined material layers, in a schematic illustration;

FIG. 2a shows a secretion-conducting, channel-like formation as it develops, within the scope of residually dimensioned sealing and/or tamponading balloon bodies, in a lumen that is smaller relative to the balloon, in a transversal section;

FIG. 2b shows a corresponding secretion-conducting, channel-like formation that enlarges in a droplet shape under reduced filling pressure, and that opens into a U shape upon further reduction of the filling pressure;

FIG. 3 shows a tracheal tube cuff, wherein the channel-like formations bridge the balloon body from one end-face side to the other, in a schematic illustration;

FIG. 4 shows a two-layer embodiment of a balloon wall, the supporting PUR layer being combined with a water vapor-tight barrier layer made of PVDC;

FIG. 5 shows a three-layer embodiment of a balloon wall, the supporting PUR layer being combined with a middle barrier layer made of PVDC and/or EVOH, and a PVC layer that attenuates the elastic straightening properties of PUR; and

FIG. 6 shows a qualitative comparison of two types of balloons formed from elastic PUR, one of the design types being combined with a layer of PVC that modifies the elastic properties of PUR.

### DETAILED DESCRIPTION OF THE PREFERRED EMBODIMENTS

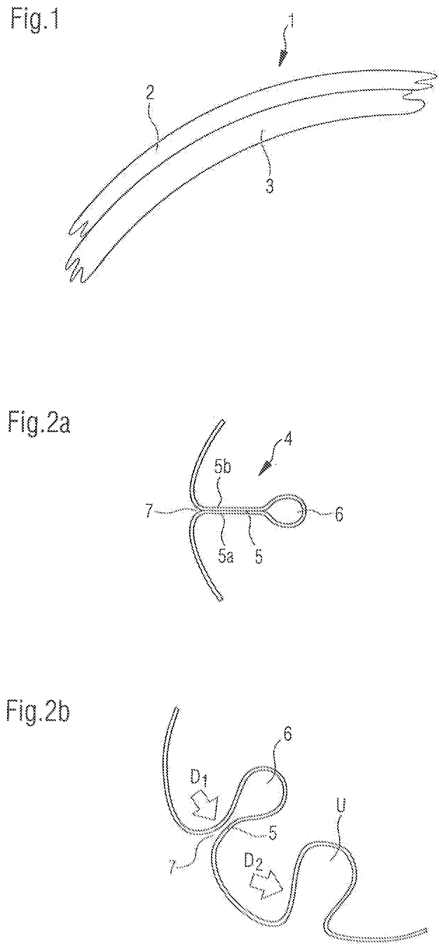

FIG. 1 schematically shows an example of a two-layer structure of a balloon wall 1 according to the invention, the outer material layer 2 that faces the particular lumen or cavity being made of Elastollan 1100 thermoplastic PUR having a Shore hardness of 90A and a proportional wall thickness of 5 to 10 microns. The material layer 3 facing the interior space of the balloon 12 is preferably made of a PVC having a Shore hardness of 70A and a proportional wall thickness of wall thickness of 15 to 20 microns. The two polymers are preferably produced flatly joined and fixedly adhered to one another directly, i.e., without an adhesion-promoting layer in between, via a coextrusion process. The PVC layer, which is 15 to 20 microns thick, on the one hand counteracts the elastic straightening of the proportional PUR layer that is folded into a loop-like formation, in a damping manner that reduces the speed and the extent of the straightening. On the other hand, the proportional PVC layer reduces the passage or the migration of polar substances through the described PUR/PVC layer combination, and thus reduces undesirable effects of condensation and accumulation of liquid, in particular water, in the interior space of the balloon.

Within the scope of the invention, the wall layers made of PVC and PUR may also be arranged inside the layer composite in such a way that the PVC layer is situated on the balloon outer side.

In addition to two-layer balloon walls, for example three-layer embodiments are also possible in which the PUR layer is preferably “sandwiched” between two PVC layers. For an overall wall thickness of 30 microns, the distribution of the individual layers may have, for example, a 12-μm PVC layer on the outside, a 6-μm PUR layer in the middle, and a 12-μm PVC layer on the outside. This embodiment is particularly advantageous in limiting undesirable migration effects of polar substances such as water.

FIG. 2a shows a diagram of the transversal section of a secretion-conducting invagination 4 as it develops fold-like structures for a residual, i.e., oversized, sealing and/or tamponading balloon body when placed inside a lumen or space that is smaller in relation to the residually dimensioned balloon, by invagination of the excess balloon wall. In particular for cyclically changing filling pressure values within the balloon, a typical wheel spoke-like arrangement of such invaginations, pointing toward the center of the balloon, results over the course of use.

The invaginations each have a web-like, flatly closed portion 5, while a loop-like formation 6 is formed at the blind end of each invagination, pointing toward the balloon center. In the region of the loop that forms, the wall of the balloon envelope makes a 180-degree turn, thus generating a pronounced opening effect on the loop-like formation due to the elastic straightening properties of the PUR layer integrated into the wall. This results in the particular sealing effect of the balloon that is effective at a certain point in time, as a function of the size of the cross-sectional area of the particular loop-like formation, and also the greatest possible avoidance or reduction of cyclical sudden changes in diameter of the loop. Due to capillary effects on secretions that are present within the loop, small cross-sectional areas of the loop act in a flow-inhibiting manner, all the way to complete stasis of the secretion or the loop contents. The inhibiting effect of the free flow of the secretion is lost with increasing expansion or enlargement of the cross-sectional area of the loop.

In addition to the particular property with regard to elastic straightening of the balloon wall that is turned in a loop-like manner, the sealing-relevant cross-sectional area of the loop-like formation 6 is determined by the filling pressure which prevails at that moment in the balloon, and which in particular is exerted against the two wall layers 5a and 5b of the web-like portion 5 of the invagination 4 and flatly presses them together in a tightly sealing manner, an open lumen remaining in the region of the turn of the two wall layers, i.e., at the blind end of the invagination in question.

The overall wall thickness of a balloon designed according to the invention preferably should not exceed 30 μm. In the preferred design of the balloon, the ratio of the proportional wall thickness of the PUR layer to the proportional wall thickness of the PVC layer is between 1:2 and 1:4, and preferably is 1:3.

For example, for a specific layer combination described in FIG. 1, if a cyclical fluctuation of the filling pressure in the balloon, generated by the patient's own breathing, between 30 mbar and 5 mbar occurs, this results in an increase of 10% to 25%, but generally not greater than 20%, in the cross-sectional area of the loops, which determines the sealing efficiency of the balloon. The largest loop diameters, measured within a particular loop-like formation, of balloons manufactured according to the invention are approximately 30 to 120 μm, preferably approximately 40 to 80 μm, at a continuous filling pressure of 30 mbar.

For cyclical fluctuations of the balloon filling pressure of, for example, 20 changes per minute and pressure amplitudes or pressure extreme values between 30 mbar and 5 mbar, the sealing properties of the balloon according to the invention, for example in the specific use for tracheal secretion sealing, are largely maintained. Pump-like, cyclically “milking” effects, synchronously following the patient's own breathing, on the loop-like formation 6 or on the channels that form the loops, as described in the medical literature for thick-walled, single-layer PVC-based cuffs having a wall thickness of 70 to 120 microns, are absent for the most part in a tracheal tube cuff designed according to the invention.

FIG. 2b shows a loop-like formation 6 corresponding to FIG. 2a, in the state of a filling pressure situation that is reduced relative to FIG. 2a. If the filling pressure falls below a certain sealing-critical filling pressure D1, the entry region 7 at the base of the invagination 4 begins to open, and the loop-like formation 6 expands and lengthens, starting at the blind inner end of the invagination and advancing toward the outer base of the invagination. The web-like, tightly sealing segment 5 of the invagination is correspondingly shortened. Upon a further drop of the filling pressure to a value D2, the web-like segment 5 opens completely, and the invagination changes into a flatly open U shape U.

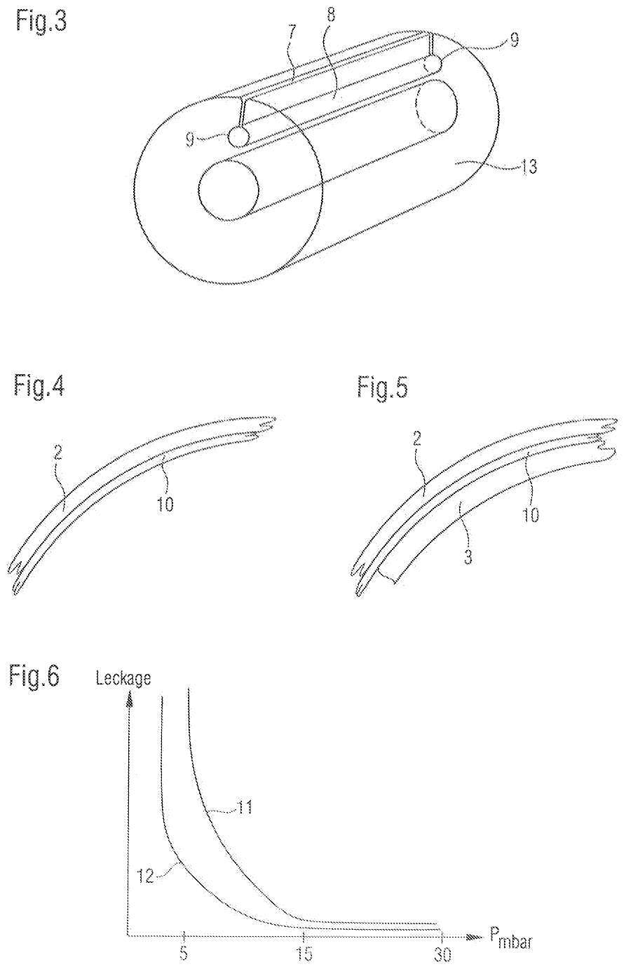

Using the example of a cylindrical sealing balloon, as used, for example, as a secretion-sealing tracheal tube cuff, FIG. 3 schematically shows channel-like formations 8 which emerge from the loop-like turned formations 6, at the blind end of the particular invaginations. The channel-like formations extend continuously from one end-face side 9 of the balloon cylinder to the opposite end-face side, and under continuous load from cyclically changing filling pressures, in many cases assume an approximately parallel alignment with respect to the cylinder axis of the balloon, thus allowing the leakage of liquids or secretions from one end-face side to the other end-face side of the balloon, which sealingly closes or tamponades in a space-filling manner a lumen or an interior space of a patient.

FIG. 4 shows a particular balloon wall having a two-layer design, the balloon-stabilizing PUR layer 2 being combined with a water vapor- and gas-tight barrier layer 10 made of PVDC. The PVDC layer 10 may be oriented toward the outer side or also toward the inner side of the balloon. PVDC has a very efficient water- and gas-sealing effect, even for very thin layer thicknesses. The proposed combination thus provides the basis for manufacturing particularly advantageous, small overall wall thicknesses of the balloon in the range of 10 to 15 microns, which are advantageous in the sense of a nonalternating cross-sectional area of the loop-like formation 6 that is as small or as constant as possible. The PUR layer 2 has a layer thickness of 5 microns, for example, whereas the PVDC layer 10 has a thickness of 5 to 10 microns, for example.

FIG. 5 shows a particular three-layer embodiment of a balloon wall, the elastic PUR layer 2 being combined with a centrally arranged gas- and water vapor-tight barrier layer 10, for example made of PVDC or alternatively EVOH, and a layer 3, preferably made of PVC having a low Shore hardness, which according to the invention attenuates the elastic straightening properties of PUR. For an overall wall thickness of 25 microns, for example, the PUR layer 2 has a layer thickness of 5 microns, for example, the gas- and water vapor-tight barrier layer 10 made of PVDC, for example, has a thickness of 5 microns, and the attenuating layer 3 made of PVC, for example, has a proportional layer thickness of 15 microns.

FIG. 6 qualitatively illustrates, based on two graphs 11, 12, how the filling pressures prevailing in the balloon interior act on the cross-sectional area of the loop-like formation 6 that is relevant for the sealing efficiency of the particular sealing or tamponading catheter or device application. In a comparative approximation, according to a graph 11 a residually dimensioned balloon that forms radial invaginations of the residual balloon envelope, and that is manufactured from a single layer of PUR having a wall thickness of 15 microns, made of “Elastollan 1190A” material, is compared to a graph 12 corresponding to a residually formed and dimensioned balloon 13 made of a two-layer material, made up of a combination according to the invention of a PUR layer and a PVC layer and having an overall wall thickness of 20 microns, as described for the technology in FIG. 1 by way of example. At approximately 20 cyclical fluctuations per minute, which in each case pass through the pressure range of 30 mbar to 15 mbar, both types of balloons have a comparably efficient sealing effect, corresponding to a virtually complete seal. However, if the lower values of the pressure fluctuations extend into the range of 15 mbar to 5 mbar, the two graphs diverge, the cross-sectional area of the loop for variant 11 being approximately 10 to 25% larger than variant 12, 13. For the single-layer balloon according to graph 11, the seal is completely lost in a pressure range below 5 mbar, which for the multilayer balloon 12, 13 according to the invention, made of a combination according to the invention of a PUR layer and a PVC layer, is not the case until below approximately 3 mbar.

### LIST OF REFERENCE NUMERALS

1 balloon wall
2 material layer
3 material layer
4 invagination
5 web-like portion
6 loop-like formation
7 entry region
8 channel-like formation
9 end-face side
10 barrier layer
11 graph
12 graph
13 balloon envelope
D1 pressure value
D2 pressure value
U U shape

1. A balloon-like structure (13) for positioning within a cavity in the human or animal body, for example within a lumen or some other interior space, in particular as an integral part of a catheter, in such a way that the stated cavity on the one hand is filled as completely as possible, i.e., without residual space, but on the other hand largely maintains its shape or is not deformed by the balloon body (13), characterized in that the balloon (13) is made of a multilayer balloon film material, at least one layer (2) being made of an elastically deformable polyurethane (PUR) and at least one other layer (3) being made of a nonelastic material such as polyvinyl chloride (PVC), wherein the at least one PUR layer (2) is made of a thermoplastic PUR of a type having a water absorption of 5% or less according to DIN ISO 62, preferably having a water absorption of 2% or less according to DIN ISO 62.

2. The balloon-like structure (13) according to claim 1, characterized in that a layer (2) made of elastically deforming PUR material is combined with a layer (3) made of plastically deforming material, in particular plastically deformable PVC.

3. The balloon-like structure (13) according to claim 1, characterized in that typical invaginations (8) of the excess residual balloon envelope, provided in the balloon interior, form upon the in situ placement of a residual balloon body, i.e., formed with excess balloon material along the circumference of the balloon.

4. The balloon-like structure (13) according to claim 3, characterized in that the invaginations (8) provided in the balloon interior have cross-sectionally loop-like turned formations (6) that preferably extend or continue as channel-like formations (9) in the longitudinal direction of the balloon (13), i.e., between its distal and proximal end-face sides (9).

5. The balloon-like structure (13) according to claim 4, characterized in that the cross-sectionally loop-like turned formations (6), which preferably extend or continue as channel-like formations (9) in the longitudinal direction of the balloon (13), have an opening diameter between 30 μm and 120 μm, preferably a loop diameter between 40 μm and 80 μm, at a filling pressure of the balloon (13) of 30 mbar.

6. The balloon-like structure (13) according to claim 5, characterized in that due to the combination with at least one layer (3) made of a nonelastic material, for example PVC, the opening diameter of the loop- or channel-like formation (6, 9) is reduced compared to the opening diameter of a loop- or channel-like formation (6, 9) for the pure PUR layer (2) of the same material type and having the same layer thickness.

7. The balloon-like structure (13) according to claim 1, characterized in that at least one layer (3) made of a nonelastic material, for example PVC, has a plastic, nonelastic quality such that its planar deformation, bending, or torsion under filling pressures of the balloon (13) that change in situ exerts an attenuating effect on the opening kinetics of loop- or channel-like formations (8).

8. The balloon-like structure (13) according to claim 1, characterized in that the elastically caused opening or expansion of the loops or channels upon a transient or cyclically fluctuating pressure drop in the balloon is slowed down due to the combination of at least one PUR layer (2) with at least one layer (3) made of a nonelastic material made of PVC, for example.

9. The balloon-like structure (13) according to claim 1, characterized in that due to the combination of at least one PUR layer (2) with at least one layer (3) made of a nonelastic material, for example PVC, the elastic straightening effect in the region of the loop- or channel-like turned formations (6, 9) is reduced in such a way that the cross-sectional areas of the secretion-conducting loop- and channel-like structures (6, 9) are reduced in contrast to a single-layer elastic balloon film (2) made only of PUR, and are also decreased in the event of cyclical fluctuations of the balloon filling pressure.

10. The balloon-like structure (13) according to claim 1, characterized in that the layer (2), instead of being made of a thermoplastic PUR of a type having a water absorption of 5% or less according to DIN ISO 62, preferably having a water absorption of 2% or less according to DIN ISO 62, is made of a material having comparable elastic properties, in particular an organic material having comparable elastic properties.

11. The balloon-like structure (13) according to claim 1, characterized in that the overall wall thickness of the balloon envelope is less than or equal to 50 μm, preferably less than or equal to 40 μm, in particular less than or equal to 30 μm.

12. The balloon-like structure (13) according to claim 1, characterized in that the ratio of the proportional wall thickness of the at least one PUR layer (2) to the proportional wall thickness of the at least one layer (3) made of a nonelastic material, for example PVC, is between 1:1 and 1:5, preferably between 1:2 and 1:4, and in particular is approximately 1:3.

13. The balloon-like structure (13) according to claim 1, characterized in that the migration of fluids, in particular polar liquids, through the wall (1) of the balloon envelope (13) is reduced due to the combination of at least one PVC layer (3) with at least one PUR layer (2).

14. The balloon-like structure (13) according to claim 1, characterized in that the loop- or channel-like turned formations (6, 9) maintain their sealing property with respect to fluids, in particular liquids, provided that the balloon filling pressure and/or the lower limit values of the allowable fluctuations of the balloon filling pressure is/are at or above 5 mbar.

15. The balloon-like structure (13) according to claim 1, characterized in that the cross-sectional areas of the loop- or channel-like turned formations (6, 9) increase by no more than 25%, preferably only by 20% or less, provided that the balloon filling pressure and/or the lower limit values of the allowable fluctuations of the balloon filling pressure is/are at or above 5 mbar.

16. The balloon-like structure (13) according to claim 4, characterized in that the cross-sectional areas of the loop- or channel-like turned formations (6, 9) increase by no more than 25%, preferably only by 20% or less, provided that the pressure amplitude and/or the difference between the two pressure extremes of the balloon filling pressure are/is in a range between 5 mbar and 30 mbar.

17. The balloon-like structure (13) according to claim 1, characterized in that at least one layer (3) is made up of a nonelastic material made of polyvinylidene chloride (PVDC).

18. The balloon-like structure (13) according to claim 1, characterized in that at least one layer (3) is made up of a nonelastic material made of ethylene vinyl alcohol (EVOH) copolymer.

19. The balloon-like structure (13) according to claim 1, characterized by a three-layer balloon envelope.

20. The balloon-like structure (13) according to claim 1, characterized in that a gas- and/or water vapor-tight barrier layer (10), preferably made of PVDC or EVOH, is situated between the elastically deformable PUR layer (2) and the nonelastically deformable layer (3) made of PVC, for example.

21. The balloon-like structure (13) according to claim 20, characterized in that the proportional wall thickness of the nonelastically deformable layer (3), made of PVC, for example, is greater than the proportional wall thicknesses of the elastically deformable PUR layer (2) and/or of the gas- and/or water vapor-tight barrier layer (10), preferably made of PVDC or EVOH.

22. The balloon-like structure (13) according to claim 20, characterized in that the ratio of the proportional wall thickness of the gas- and/or water vapor-tight barrier layer (10), preferably made of PVDC or EVOH, to the proportional wall thickness of the nonelastically deformable layer (3), for example PVC, is between 1:1 and 1:5, preferably between 1:2 and 1:4, and in particular is approximately 1:3.

23. The balloon-like structure (13) according to claim 20, characterized in that the joining of the multiple layers (2, 3, 10) of the balloon envelope (13) is brought about by coextrusion of the various layers (2, 3, 10).

24. The balloon-like structure (13) according to claim 1, characterized in that the balloon envelope (13) is manufactured by blow molding of a multilayer extruded tube blank.
